(19) United States
(12) Patent Application Publication
(10) Pub. No.: US 2017/0172419 A1
(43) Pub. Date: Jun. 22, 2017

(54) PHOTOACOUSTIC APPARATUS AND SIGNAL PROCESSING METHOD

(71) Applicant: CANON KABUSHIKI KAISHA, Tokyo (JP)

(72) Inventors: Takuji Oishi, Kawasaki-shi (JP); Kazuhito Oka, Tokyo (JP)

(21) Appl. No.: 15/116,015

(22) PCT Filed: Feb. 6, 2015
(86) PCT No.: PCT/JP2015/000548

§ 371 Date: Aug. 2, 2016

(30) Foreign Application Priority Data
Feb. 10, 2014 (JP) 2014-023285
Jan. 16, 2015 (JP) 2015-006471

(51) Int. Cl. A61B 5/00

(52) U.S. Cl. CPC A61B 5/0095; A61B 5/7203; A61B 5/7257; A61B 2576/00

(57) ABSTRACT

The present invention employs a photoacoustic apparatus including: a receiving unit that receives acoustic waves at a plurality of measurement positions and converts the acoustic waves to a plurality of time-series reception signals; and a processing unit that adjusts phases of the reception signals so that a phase pattern of a target signal among the reception signals corresponds to a specific spatial direction, reduces low-frequency components in the specific spatial direction from the reception signals of which the phases have been adjusted, and acquires characteristic information of an object based on the reception signals of which the low-frequency components have been reduced.

### TECHNICAL FIELD

The present invention relates to a photoacoustic apparatus and a signal processing method.

### BACKGROUND ART

In recent years, in a medical field, photoacoustic tomography (PAT) which obtains biological functional information using light and ultrasonic waves has been proposed and developed as one of apparatuses that image the inside of a living body in a non-invasive manner.

Photoacoustic tomography is a technique of imaging tissues inside a living body, serving as an acoustic wave generation source using a photoacoustic effect.

Photoacoustic effect is a phenomenon that, when an object is irradiated with a pulsating beam generated from a light source, light having propagated and diffused inside the object is absorbed, whereby acoustic waves (typically ultrasonic waves) are generated. A change over time in the received acoustic waves is detected at a plurality of positions to obtain signals, the obtained signals are mathematically analyzed, that is, reconstructed, and information related to optical characteristics such as an absorption coefficient inside the object is visualized three-dimensionally.

When near-infrared rays are used as the pulsating beam, since the near-infrared rays easily pass through water which constitutes a major part of a living body and are easily absorbed by hemoglobin in the blood, it is possible to image blood vessels. Further, by comparing blood vessel images associated with pulsating beams of different wavelengths, it is expected that an oxygen saturation in the blood which is functional information can be measured. That is, since it is thought that the blood around a malignant tumor has a lower oxygen saturation than the blood around a benign tumor, it is possible to distinguish a benign tumor from a malignant tumor based on the oxygen saturation.

Moreover, an ultrasonic examination apparatus is an example of an apparatus that receives acoustic waves to image biological functional information similarly to photoacoustic tomography. The ultrasonic examination apparatus transmits acoustic waves to a living body, receives acoustic waves reflected inside the living body, and images the reflected acoustic waves. Acoustic waves have such properties that the acoustic waves reflect from an interface where the acoustic impedance which is the product of a propagation velocity and the density of acoustic waves changes. Thus, the ultrasonic examination apparatus can visualize a distribution of acoustic impedances in a living body.

In the photoacoustic tomography and the ultrasonic examination, an image reflecting the characteristic information of a living body is obtained by imaging received acoustic waves. However, in this case, artifacts which are not present actually appear, which may sometimes disturb diagnosis. Although various causes for artifacts are known, one of the causes is an acoustic artifact which appears when an acoustic wave is actually generated at an unexpected position and is reflected.

For example, when an acoustic wave is reflected in the course of propagating to an acoustic detector and is received later than the point in time when the acoustic wave is directly received by the acoustic detector, an acoustic artifact associated with the reflection appears at a position away from the acoustic detector further than the position where an object is actually present. Besides this, an acoustic artifact may appear when an acoustic wave is generated from an apparatus housing and is received at the same time as an acoustic wave generated within an object.

Acoustic artifacts can be weakened by modifying a propagation path of the acoustic wave. For example, it is preferable to remove non-target light absorbers (light absorbers other when a reflection layer or an object when the reflection layer, the object, or the like is an imaging target) away from the propagation path of the acoustic wave. However, due to apparatus limitations, it may not be possible to remove the non-target light absorbers away from the propagation path.

In such a case, acoustic artifacts may be reduced by software-based processing. Since acoustic artifacts are generated when acoustic waves are received actually, an acoustic artifact signal is mixed at the point in time when signals are received. However, if the propagation path of acoustic waves is known, it is possible to identify the acoustic artifact signal within the obtained signals.

### CITATION LIST

### Patent Literature

### [PTL 1]

Japanese Patent Application Laid-Open No. 2011-217767

### SUMMARY OF INVENTION

### Technical Problem

However, conventionally, even if the acoustic artifact signal is known, since the signal is superimposed on signals from the inside of the object, it is difficult to separate and reduce the acoustic artifact signal only.

The acoustic artifact signal may be reduced by using optimization. However, this optimization incurs long calculation time and has limitations on the apparatus size and cost. Further, it is difficult to reduce the acoustic artifact signal satisfactorily when the parameters such as the speed of sound and the model used for the optimization are different from the actual parameters and model and when the acoustic artifact signal exhibits little difference from the signals obtained from the inside of the object.

Moreover, as disclosed in PTL 1, the acoustic artifact signal can be reduced when signals are projected onto a separate space using Fourier transform or the like and the acoustic artifact signal can be separated from the signals obtained from the inside of the object. However, the conditions for being able to separate artifacts in such a separate space are limited. In fact, since the acoustic artifact signals are received at various points in time and have various intensities, it may be difficult to apply this technique to an optional case.

The present invention has been made based on recognition of such problems. An object of the present invention is to provide a technique of acquiring characteristic information of an object using acoustic waves while separating artifacts superimposed on signals to reduce the influence thereof.

### Solution to Problem

The present invention provides a photoacoustic apparatus comprising:

a receiving unit configured to receive acoustic waves at a plurality of measurement positions and convert the acoustic waves to a plurality of time-series reception signals; and

a processing unit configured to, (a) adjust phases of the plurality of time-series reception signals so that a phase pattern of a target signal among the plurality of time-series reception signals corresponds to a specific spatial direction, (b) reduce low-frequency components in the specific spatial direction from the plurality of time-series reception signals of which the phases have been adjusted, and (c) acquire characteristic information of an object based on the plurality of time-series reception signals of which the low-frequency components have been reduced.

The present invention also provides a photoacoustic apparatus comprising:

a receiving unit configured to receive acoustic waves at a plurality of measurement positions and convert the acoustic waves to a plurality of time-series reception signals; and

a processing unit configured to, (a) acquire information on an arrangement direction of a target signal among the plurality of time-series reception signals, (b) reduce low-frequency components in the arrangement direction of the target signal from the plurality of time-series reception signals based on the information on the arrangement direction of the target signal, and (c) acquire characteristic information on an inside of an object based on the plurality of time-series reception signals of which the low-frequency components have been reduced.

The present invention also provides a signal processing method of acquiring characteristic information of an object based on a plurality of time-series reception signals obtained by receiving acoustic waves at a plurality of measurement positions, comprising:

adjusting phases of the plurality of time-series reception signals so that a phase pattern of a target signal among the plurality of time-series reception signals corresponds to a specific spatial direction;

reducing low-frequency components in the specific spatial direction from the plurality of time-series reception signals of which the phases have been adjusted; and

acquiring characteristic information of an object based on the plurality of time-series reception signals of which the low-frequency components have been reduced.

The present invention also provides a signal processing method of acquiring characteristic information of an object based on a plurality of time-series reception signals obtained by receiving acoustic waves at a plurality of measurement positions, comprising:

acquiring information on an arrangement direction of a target signal among the plurality of time-series reception signals;

reducing low-frequency components in the arrangement direction of the target signal from the plurality of time-series reception signals based on the information on the arrangement direction of the target signal; and

acquiring characteristic information of an inside of an object based on the plurality of time-series reception signals of which the low-frequency components have been reduced.

### Advantageous Effects of Invention

According to the present invention, it is possible to provide a technique of acquiring characteristic information of an object using acoustic waves while separating artifacts superimposed on signals to reduce the influence thereof.

Further features of the present invention will become apparent from the following description of exemplary embodiments with reference to the attached drawings.

### BRIEF DESCRIPTION OF DRAWINGS

FIG. 1 is a schematic diagram illustrating the arrangement of an apparatus according to an embodiment of the present invention.

FIGS. 2A and 2B are schematic diagrams for describing signals generated by the apparatus according to the embodiment of the present invention.

FIGS. 3A to 3D are schematic diagrams for describing processing of the apparatus according to the embodiment of the present invention.

FIG. 4 is a schematic diagram illustrating the configuration of the apparatus according to the embodiment of the present invention.

FIG. 5 is a schematic diagram illustrating an implementation method of an apparatus according to an embodiment of the present invention.

FIGS. 6A and 6B are schematic diagrams for describing signals generated by the apparatus according to an embodiment of the present invention.

FIG. 7 is a schematic diagram illustrating the configuration of the apparatus according to the embodiment of the present invention.

FIG. 8 is a schematic diagram illustrating the configuration of the apparatus according to the embodiment of the present invention.

FIG. 9 is a schematic diagram illustrating the arrangement of an apparatus according to an embodiment of the present invention.

FIG. 10 is a schematic diagram illustrating signals generated by the apparatus according to the embodiment of the present invention.

FIGS. 11A and 11B are schematic diagrams illustrating processing of the apparatus according to the embodiment of the present invention.

FIG. 12 is a schematic diagram illustrating the configuration of the apparatus according to the embodiment of the present invention.

FIG. 13 is a schematic diagram illustrating the arrangement of the apparatus according to the embodiment of the present invention.

FIGS. 14A and 14B are diagrams illustrating the processing results of the apparatus according to the embodiment of the present invention.

FIGS. 15A to 15D are diagrams illustrating the processing results of the apparatus according to the embodiment of the present invention.

FIGS. 16A and 16B are schematic diagrams for describing signals generated by the apparatus according to an embodiment of the present invention.

### DESCRIPTION OF EMBODIMENTS

Hereinafter, a preferred embodiment of the present invention will be described with reference to the drawings. The dimensions, materials, shapes and relative positions, and the like of the constituent components described below should be changed appropriately depending on the configuration and various conditions of the apparatus to which the invention is applied, and it is not intended to limit the scope of the invention to the description given below.

The present invention relates to a technique of detecting acoustic waves propagating from an object to generate and acquire characteristic information on the inside of the object. Thus, the present invention can be understood as an acoustic wave measurement apparatus or a control method thereof, or an acoustic wave measurement method and a signal processing method and can be understood as an object information acquiring apparatus or a control method thereof, or an object information acquisition method. Further, the present invention can be understood as a program for causing an information processing apparatus having hardware resources such as a CPU to execute these methods and a storage medium storing the program.

An object information acquiring apparatus of the present invention includes an apparatus which uses a photoacoustic tomography technique of irradiating an object with light (electromagnetic waves) and receiving (detecting) acoustic waves generated and propagated at specific positions inside the object or on the object surface. Such an object information acquiring apparatus can be also referred to as a photoacoustic apparatus because the apparatus obtains characteristic information on the inside of the object based on photoacoustic measurement in the form of image data or the like.

The characteristic information in the photoacoustic apparatus represents a generation source distribution of acoustic waves generated by light irradiation, an initial acoustic pressure distribution inside an object, or a light energy absorption density distribution and an absorption coefficient distribution derived from the initial acoustic pressure distribution, and a density distribution of materials that constitute tissues. Examples of the materials that constitute tissues includes blood components such as an oxygen saturation distribution or a redox hemoglobin density distribution, or fat, collagen, and water.

The object information acquiring apparatus of the present invention includes an ultrasonic apparatus that transmits acoustic waves to an object, receives reflection waves (echo waves) reflected from specific positions inside the object, and obtains characteristic information in the form of image data or the like. The characteristic information in the ultrasonic apparatus is information that reflects surface shape based on reflection waves at positions where the acoustic impedances of a tissue inside the object are different.

The acoustic waves referred to in the present invention are typically ultrasonic waves and include elastic waves called sound waves and acoustic waves. The acoustic waves generated by a photoacoustic effect are referred to as photoacoustic waves or photoultrasonic waves. Electrical signals converted from acoustic waves by a probe are also referred to as acoustic signals.

### First Embodiment

Embodiments of the present invention will be described. The present invention adjusts the delay of respective acoustic signals received at a plurality of measurement positions so that separation target signals have the same phase, separates and reduces the in-phase signals, restores the delay to an original one to thereby separate and reduce separation target signals. Even when it is not possible to remove target signals completely, it is possible to reduce artifacts by reducing the target signals.

Moreover, the present embodiment is implemented based on photoacoustic tomography. However, the same technique can be applied to an ultrasonic examination apparatus. First, the principle of the present invention and the present embodiment will be described, and then, constituent components and an implementation method will be described, followed by the effects lastly.

(Principle of Delay of Acoustic Wave)

In order to explain the principle of the present invention, signals in photoacoustic tomography will be described. In the present embodiment, reflection signals are used as separation target signals. However, the present invention is not limited to the reflection signals, an optional signal that a user wants to separate may be used.

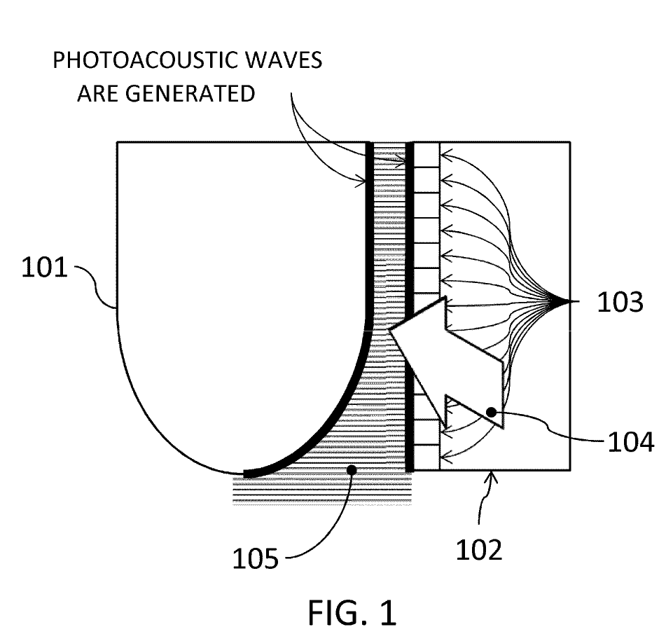

In FIG. 1, a plurality of acoustic detection devices 103 included in an acoustic detector 102 receives photoacoustic waves generated and propagated from an object 101 irradiated with a pulsating beam 104 with an acoustic matching member 105 interposed.

Only one acoustic detection device may be provided. In this case, a scanning mechanism that moves a measurement position on the object, of the acoustic detection device may be provided so that photoacoustic waves can be detected at a plurality of measurement positions. When the technique of a reduction process according to the present invention is applied to acoustic signals obtained at respective measurement positions, the same effects as respective embodiments are obtained.

In photoacoustic tomography, at positions where pulsating beams emitted from a light source are absorbed, acoustic waves corresponding to the amount of absorption are generated. As illustrated in FIG. 1, since the surface of the object 101 and the surface of the acoustic detector 102 are irradiated with a strong pulsating beam that is not decayed, strong acoustic waves are generated. The propagation direction of the generated acoustic waves is normal to the object surface and the acoustic detector surface.

The acoustic waves generated from the object surface and the acoustic detector surface propagate through the acoustic matching member 105 and reach the acoustic detector surface and the object surface, respectively. Some components pass and propagate as they are and the remaining components are reflected. The proportion of transmitted and reflected components depends on acoustic impedances of respective materials. Acoustic waves are reflected so that an incidence angle is equal to a reflection angle similarly to light.

Since the velocity of a pulsating beam is sufficiently faster than acoustic waves, it can be considered that the acoustic waves occur at the same time regardless of the occurrence position. Thus, a signal obtained when a signal generated from the object surface reaches first the acoustic detector is delayed by the amount corresponding to the thickness of the acoustic matching member. Here, the thickness of the acoustic matching member means the thickness of the acoustic matching member in the time direction viewed from the acoustic detector.

Moreover, since a signal obtained when an acoustic wave generated from the acoustic detector surface returns after being reflected from the object surface has passed through the acoustic matching member twice, the signal is also delayed by the amount corresponding to the thickness of the acoustic matching member. When reflection is repeated further, a delay corresponding to the thickness of the acoustic matching member occurs.

Since the thickness of the acoustic matching member is determined by the shape of the object surface and the arrangement of probe devices (that is, the shape of the acoustic detector), the delay of acoustic waves associated with reflection can be estimated from the shape of the object surface and the shape of the probe. That is, it is possible to calculate the delay time as long as the distance of a propagation path and the sound velocity in a medium through which the acoustic wave passes are known. The same can be said to be true when the shape of the acoustic detector is not planar. The delay time indicates a difference between the arrival points in time when the phase differences of signals derived from acoustic waves which propagate from a specific position to respective devices are adjusted so that the temporal origins are aligned.

(Difference in Delay Amount Depending on Distance)

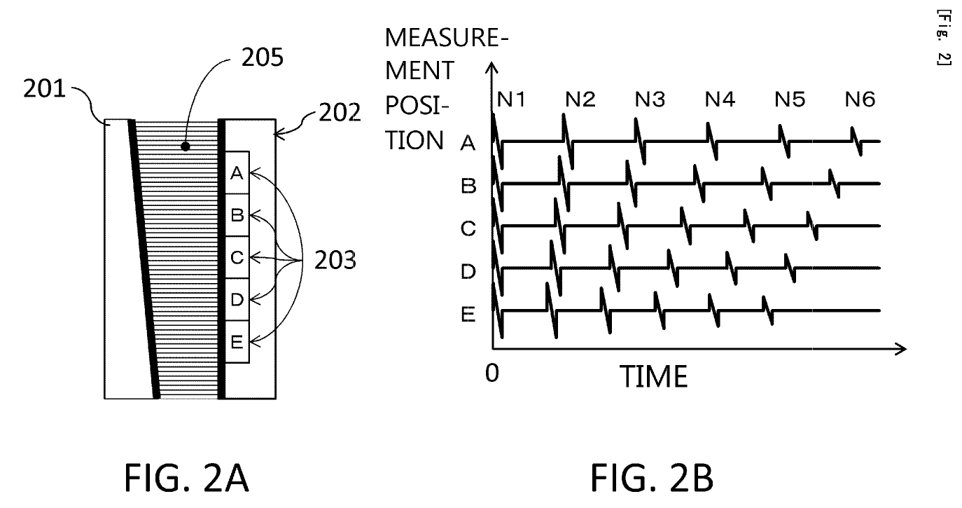

As illustrated in FIG. 2A, a case where the acoustic detector surface is slightly inclined with respect to the object surface will be considered. In FIG. 2A, an object 201 and an acoustic detector 202 including acoustic detection devices 203 (A to E) are in contact with each other with an acoustic matching member 205 interposed.

FIG. 2B illustrates signals obtained by the acoustic detection devices 203 (A to E) in FIG. 2A, in which the device positions are identical in FIGS. 2A and 2B. Moreover, the vertical axis of respective signals represents a voltage and shows the intensity of a photoacoustic wave. Moreover, the horizontal axis represents time and the point in time when light is emitted is the origin 0.

In FIG. 2B, a signal indicated by N1 is a signal generated from the acoustic detector surface and the point in time for each signal is identical. A signal N2 is a signal detected when the signal generated from the object surface reaches the acoustic detector. A signal N3 is a signal detected when an acoustic wave propagated toward the object among the acoustic waves generated from the acoustic detector surface is reflected from the object surface and returns to the acoustic detector. A signal N4 is a signal detected by the acoustic detector when an acoustic wave generated from the object surface propagates up to the acoustic detector and is then reflected from the acoustic detector and is further reflected from the object surface. Similarly, acoustic waves generated from the acoustic detector surface and the object surface undergo multiple reflection and are detected as signals N5, N6, . . . , and so on.

In this manner, when the acoustic detector surface and the object surface are inclined with respect to each other, a difference in the arrival time of acoustic waves occurs depending on a propagation path length and the difference increases as the number of reflections increases. Moreover, the detected intensity decreases gradually.

(Principle of Signal Separation)

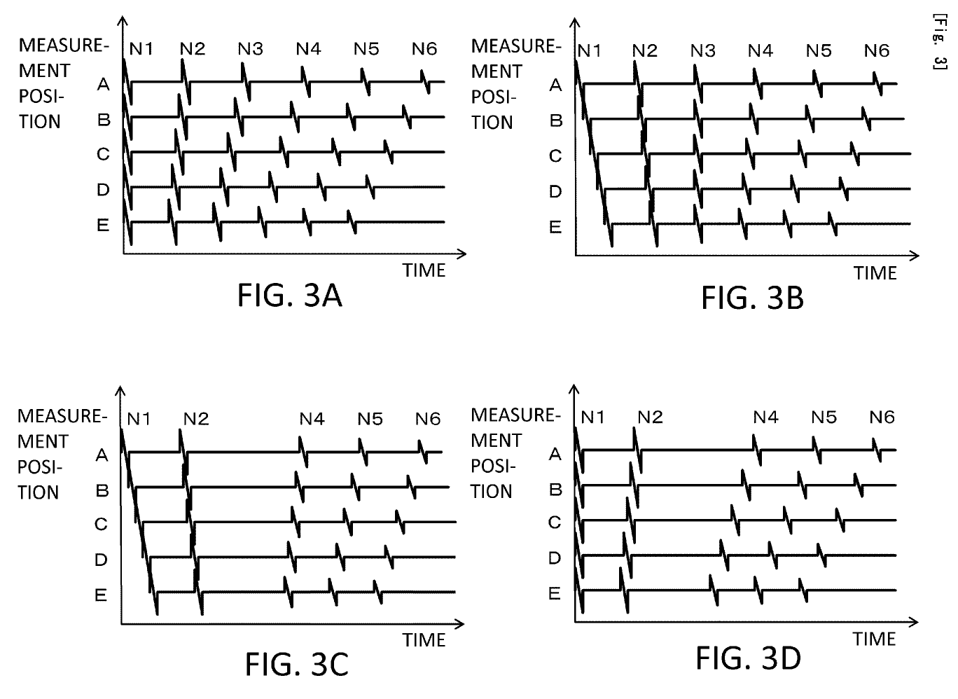

Next, the principle of the present invention will be described. FIG. 3A illustrates signals obtained using the system of FIG. 2A. In this example, it is assumed that the signal N3 which is a reflection signal is a separation target signal. Moreover, a relation between the positions (measurement positions) of the acoustic detection devices as illustrated in FIG. 3A and a relative delay time of signals obtained by the respective acoustic detection devices will be referred to as a delay profile.

In the present invention, first, the delay profiles of separation target signals are acquired, and as illustrated in FIG. 3B, the delays of respective obtained reception signals are adjusted so that the separation target signals appear at the same point in time (that is, the signals have the same phase). When signals are arranged so that the temporal origins form a straight line or a flat surface, and the signals are observed in the arrangement direction, in-phase signals are low-frequency components of which the signal intensities change smoothly or rarely. On the other hand, signals of which the phase differences have not been adjusted include high-frequency components since the signal intensities are different depending on the measurement position and change abruptly. In this manner, when the phases of target signals are aligned in the arrangement direction of a plurality of measurement positions, signal processing is made easy. However, as will be described later, it is not essential to align phases in this manner.

Thus, by separating components having a low spatial frequency in the arrangement direction and components having a high spatial frequency, it is possible to separate in-phase separation target signals.

FIG. 3C illustrates signals obtained by extracting components having a high spatial frequency in the arrangement direction. Further, when the delay adjusted for the reflection signals of the respective signals to have the same phase is restored to the original delay, signals in which the separation target signals are separated are obtained as illustrated in FIG. 3D.

According to this principle, even when other signals are superimposed on the reflection signals, by projecting the signals onto a frequency space, it is possible to separate the reflection signals and the other signals. Moreover, since it is possible to shift optional signals to a low-frequency region in a frequency space by aligning the phases of separation target signals, this technique can be broadly applied to various separation target signals.

In the present invention, although signals are preferably arranged in such a manner that the temporal origins forma straight line or a flat surface, the signals may be arranged so that the temporal origins forma circle, a spherical surface, or the like. In this case, when separation target signals have the same phase, since the separation target signals are arranged on one circular arc or a spherical surface, it is possible to separate the separation target signals by separating intensity frequency components of signals arranged every radius of a circle or a sphere. Further, the signals may be arranged so that the temporal origins forma curved line or a curved surface.

(Apparatus Configuration)

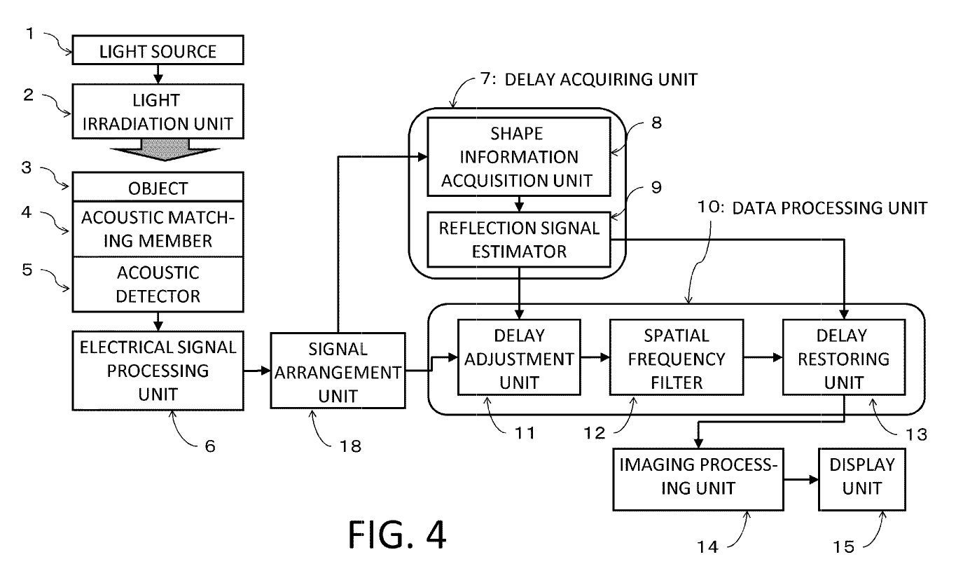

Next, constituent components of the present invention will be described with reference to FIG. 4. The object information acquiring apparatus of the present invention includes a light source 1, a light irradiation unit 2, an acoustic matching member 4, an acoustic detector 5, an electrical signal processing unit 6, a signal arrangement unit 18, a delay acquiring unit 7, a data processing unit 10, an imaging processing unit 14, and a display unit 15. Moreover, a measurement target of the present invention is an object 3.

The delay acquiring unit 7 includes a surface shape acquisition unit 8, and a reflection signal estimator 9. The data processing unit 10 includes a delay adjustment unit 11, a spatial frequency filter 12, and a delay restoring unit 13. In the following description, a reflection signal is a separation target signal.

(Light Source)

The light source 1 is a device that generates a pulsating beam. In order to obtain a large output, a laser is preferred as the light source, and a light-emitting diode or the like may also be used. In order to generate photoacoustic waves efficiently, it is necessary to emit light in a sufficiently short period according to thermal properties of an object. When the object is a living body, the pulse width of a pulsating beam generated by the light source is preferably several tens of nanoseconds or shorter.

Moreover, the wavelength of the pulsating beam is in a near-infrared region called a biological window and is preferably in the range of approximately 700 nm to 1200 nm. Light in this region is preferable to obtain information on the deep part since it reaches relatively a deep part of a living body. Further, the wavelength of the pulsating beam preferably has a high absorption coefficient with respect to an observation target.

(Light Irradiation Unit)

The light irradiation unit 2 is a device that guides the pulsating beam generated by the light source 1 to the object 3. Specifically, the light irradiation unit 2 is an optical device such as an optical fiber, a lens, a mirror, and a diffuser. These optical devices are used for changing irradiation conditions such as an irradiation shape of a pulsating beam, an optical density, or an irradiation direction in which the object is irradiated with light. These conditions may be adjusted by the light source 1. Moreover, in order to acquire a wide range of data, the light irradiation unit 2 may be moved for scanning so that the irradiation position of the pulsating beam is scanned. In this case, it is preferable to perform scanning in synchronization with the acoustic detector 5. Optical devices other than the optical devices mentioned above can be used as long as the devices have the above-described functions.

(Object)

The object 3 is a measurement target. Examples of the object 3 include a living body or a phantom that simulates the acoustic and optical properties of the living body. A photoacoustic diagnosis apparatus can image a light absorber having a large absorption coefficient present inside the object 3.

In the case of living bodies, examples of an imaging target include hemoglobin, water, melanin, collagen, and fat. In the case of phantoms, a material that simulates the optical properties of such an imaging target is enclosed in a phantom as a light absorber. Moreover, the shape and properties of a living body changes from person to person and from sample to sample. Further, a living body or a phantom in which a contrast agent, a molecule probe, or the like is injected may be used as the object.

(Acoustic Matching Member)

The acoustic matching member 4 is provided between the object 3 and the acoustic detector 5 so as to couple both acoustically so that acoustic waves can easily propagate. The acoustic matching member 4 is provided between the object 3 and the acoustic detector 5 so as to couple the two acoustically so that acoustic waves can easily propagate from the object 3 to the acoustic detector (however, it is practically impossible to completely prevent the occurrence of reflections). In this way, it is possible to prevent photoacoustic waves from being generated from the acoustic matching member to appear as artifacts on an image and to irradiate the object with a large amount of light. Moreover, the acoustic matching member is preferably uniform. An acoustic matching GEL, water, oil, and the like are used as the acoustic matching member.

(Acoustic Detector)

The acoustic detector 5 includes at least one acoustic detection device that converts acoustic waves into electrical signals. In photoacoustic tomography, acoustic waves are received from a plurality of positions to perform three-dimensional imaging. Due to this, one acoustic detection device is moved to a plurality of positions for scanning, or a plurality of acoustic detection devices is provided at different positions to receive acoustic waves from a plurality of positions. The acoustic detector 5 preferably has a high sensitivity and a broad frequency range, and specifically, acoustic detectors which use PZT, PVDF, cMUT, and a Fabry-Perot interferometer can be used. Other acoustic detectors other than the detectors mentioned above can be used as long as the detectors have the above-described functions. The acoustic detector corresponds to a receiving unit according to the present invention.

(Electrical Signal Processing Unit)

The electrical signal processing unit 6 amplifies electrical signals obtained by the acoustic detector 5 and converts the same into digital signals. A specific example of the electrical signal processing unit 6 includes an amplifier, an analog-digital converter (ADC), and the like formed of electric circuits. In order to acquire data efficiently, preferably, the same number of amplifiers and ADCs as the number of detection devices of the acoustic detector 5 are provided. However, one amplifier and one ADC may be sequentially connected and used.

The electrical signal processing unit and a signal arrangement unit, a delay acquiring unit, a data processing unit, and an imaging processing unit described later correspond to a processing unit according to the present invention. The processing unit is configured to be capable of realizing at least a portion of the functions of these respective units. The processing unit can be realized as an information processing apparatus or a processing circuit that operates according to a program.

(Signal Arrangement Unit)

The signal arrangement unit 18 is a device that receives digital signals obtained by the electrical signal processing unit 6 and arranges the digital signals on a memory inside the signal arrangement unit 18. Specifically, since the received digital signals are arranged such that the signals of all devices are arranged in a line on the memory, the signal arrangement unit 18 separates the signals of all devices stored in the memory into signals of respective devices and rearranges the signals in a desired arrangement. Since the arrangement of signals is an imaginary arrangement on the memory, the signal arrangement unit 18 may rearrange the signals in a desired arrangement by adjusting the addresses of the memory in which the signals obtained by the electrical signal processing unit 6 are stored.

The signal arrangement unit 18 can arrange the signals so as to be aligned to the same vector of a space in which the temporal origins of the signals form a straight line or a flat surface, and the time directions of the signals are divided by the line or the plane formed by the temporal origins. In this case, it is preferable to arrange the temporal origins of the signals on a plane on which a spatial arrangement of actual acoustic detection devices is projected.

Moreover, the signals may be arranged so that the temporal origins of the signals form a circle, a spherical surface, a curved line, or a curved surface. Moreover, the arranged positions may be exchanged. Moreover, signal arrangement may not be performed at this stage but may be performed during the processing of the subsequent delay acquiring unit 7 or the subsequent data processing unit 10

When the signals are arranged in this manner, since the user can easily understand the arrangement when designating the separation target signals as will be described in the second embodiment, the operation is simplified. However, the signals may not be rearranged as long as it is possible to designate the separation target signals.

(Delay Acquiring Unit)

The delay acquiring unit 7 obtains a delay profile of acoustic waves reflected from the acoustic matching member 4. The delay acquiring unit 7 includes the surface shape acquisition unit 8 and the reflection signal estimator 9. In the present embodiment, the delay acquiring unit acquires the delay profile of the reflection signal using the surface shape of the object. However, the delay acquiring unit is not limited to this but may use optional information as long as it is possible to acquire the delay profile. When a plurality of separation target signals is present, a plurality of delay profiles is obtained.

(Shape Information Acquisition Unit)

The surface shape acquisition unit 8 acquires surface shape of the object 3 included in a reception region of the acoustic detector 5. When the acoustic detector 5 scans two-dimensionally to acquire three-dimensional data including time, the acquired surface shape of the object 3 needs to be a three-dimensional shape. When the acoustic detector 5 acquires two-dimensional data, although it is sufficient that the surface shape of the object 3 is two-dimensional so as to conform with the acoustic detector 5, it is preferable to acquire a three-dimensional surface shape in order to improve accuracy.

The surface shape of the object 3 may be obtained from photoacoustic signals, and alternatively, the same can be obtained using a camera capable of measuring stereoscopic information or a laser range finder or by irradiation of ultrasonic waves. In the present embodiment, a method of obtaining the surface shape from photoacoustic signals (electrical signals originating from photoacoustic waves) will be described in detail. When the surface shape of the object 3 is obtained from photoacoustic signals, it is possible to obtain the surface shape of the object 3 without introducing a new device.

Moreover, the surface shape acquisition unit 8 may acquire surface shape by reading surface shape corresponding to the shape of an object during measurement from a plurality of pieces of surface shape stored in advance in the surface shape acquisition unit 8. In this case, a user may input the shape of an object during measurement and the type or the like of a member that holds the object with the aid of an input unit and the surface shape acquisition unit 8 may read the surface shape of the object corresponding to the input data. Alternatively, the surface shape acquisition unit 8 may detect the type of a member that holds an object and read the surface shape of the object corresponding to the detected member type.

A specific processing method of this technique will be described. Although it is possible to obtain a strong acoustic wave from the surface shape of the object 3, it is not possible to obtain a strong acoustic wave from the acoustic matching member located closer to the acoustic detector. Further, since the signals obtained from the surface of the acoustic detector appear at the same time regardless of the object, it is possible to easily specify the signals based on the points in time when intensity peaks appear. Thus, an appropriate threshold may be provided for the obtained signals, and the earliest signal other than the surface signal of the acoustic detector among the signals equal to or higher than the threshold may be determined to be the surface signal of the object. When the time at which the surface signal appears is obtained, it is possible to acquire the time corresponding to the distance from the acoustic detector to the object surface.

Since this time is the time taken for an acoustic wave to propagate from the object surface to the acoustic detector, it is possible to calculate the distance to the object surface by using the propagation velocity of the acoustic wave in the acoustic matching member. As a result, it is possible to acquire the surface shape.

In this manner, by acquiring the shape of the acoustic matching member and the object surface using the delay acquiring unit, it is possible to acquire the arrangement of reduction target signals which cause artifacts (that is, the phase pattern in the spatial direction).

When the surface shape of the object is obtained by a camera or a laser range finder, a spatial distance is converted to time. Since signals are already arranged by the signal arrangement unit 18, the time corresponding to the surface shape of the object on the arranged signals is referred to the delay profile. When the signals are not arranged by the signal arrangement unit 18, the signals are arranged at this stage to obtain the delay profile.

In this case, preferably, processes such as noise reduction, reduction in the in-phase components of the plurality of signals, or template matching may be applied to the signals to enhance the signals from the object surface. In this way, robustness of the process is improved. Moreover, although it is preferable to automatically acquire the surface shape of the object 3 from signals, a user may manually designate the surface shape by judgment based on the signals.

(Reflection Signal Estimator)

The reflection signal estimator 9 acquires the shape of the acoustic matching member from the surface shape of the object and the shape of the acoustic detector obtained by the surface shape acquisition unit 8 and acquires a delay profile of the reflection signal reflected from both interfaces of the acoustic matching member.

The reflection signal estimator 9 obtains the delay profile of the reflection signal by taking advantage of the fact that the delay profile of the reflection signal reflected from the acoustic matching member can be approximated to a delay profile obtained by delaying the delay profile of the surface shape of the object in the time direction in an integer multiple. Specifically, the process of delaying the delay profile of the surface shape of the object in the time direction by an integer multiple is a process of multiplying the delay time of the signals at the respective measurement positions forming the delay profile of the surface shape of the object by an integer. A relative relation of the respective delay times obtained as the result of the process is the delay profile of the reflection signal obtained by delaying the delay profile of the surface shape of the object in the time direction by an integer multiple.

Moreover, a reflection wave reflected from an interface of the acoustic matching member is further reflected from the opposite interface. Such a repetition of reflection is referred to as multiple reflection. Theoretically, multiple reflections continue endlessly. However, since reflection waves are decayed every time reflection occurs, if the reflection waves are sufficiently decayed as compared to a signal to be measured, the subsequent multiple-reflections may be ignored. Thus, it is preferable to determine the number of delay profiles of a reflection signal to be estimated according to the number of reflections when the reflection signal is sufficiently decayed.

The number of delay profiles of a reflection signal to be estimated is determined in advance and is preferably stored in the reflection signal estimator 9 or a storage unit. In this way, it is possible to reduce the user's operations. Moreover, the user may designate the number of delay profiles for each measurement. In this way, even when decay of reflections is different from object to object, it is possible to execute an appropriate amount of processing.

The number of delay profiles may be determined based on the size of an object and the propagation period of a reflection wave and may be determined based on the number of reflections when the reflection wave becomes sufficiently small. When the determined number of delay profiles of a reflection signal to be estimated is M, and the delay profile of a signal indicating the object surface is extended twice, three times, . . . , and M times in the time direction, the delay profiles of (M−1) reflection signals are obtained.

Moreover, in the present embodiment, although all multiple-reflection signals up to a designated number of times are target signals to be separated and reduced, only reflection signals which have been reflected a certain number of times may be separated and reduced. Moreover, in this example, although the delay profile of the surface shape of the object is delayed in the time direction by an integer multiple to obtain the delay profile of the reflection signal, propagation of acoustic waves may be simulated using the shape of the object and the shape of the acoustic detector to obtain the delay profile.

(Data Processing Unit)

The data processing unit 10 as a signal processing unit separates and reduces the reflection signal using the method described in connection with the principle based on the obtained delay profile of the reflection signal. When a plurality of separation target signals is present, a plurality of processes is performed in such a way that the process is performed using one delay profile to obtain an output, and the same process is performed on the output using another delay profile. In the present embodiment, the data processing unit 10 includes the delay adjustment unit 11, the spatial frequency filter 12, and the delay restoring unit 13.

(Delay Adjustment Unit)

The delay adjustment unit 11 adjusts the delays of the obtained digital signals at respective measurement positions based on the delay profile of the reflection signal estimated by the delay acquiring unit 7 so that the reflection signals at all measurement positions are delayed at the same time. In this way, the signals having the same delay profile as the delay profile of the reflection signal have the same delay (the same phase). This signal will be referred to as a delay adjustment signal. In the present embodiment, although the signals are already arranged since the signal arrangement unit 18 is on the preceding stage, signal arrangement may be not performed in the delay adjustment unit 11. The delay adjustment unit corresponds to a phase adjustment unit according to the present invention.

(Spatial Frequency Filter)

The spatial frequency filter 12 reduces components having a low spatial frequency in the arrangement direction of the temporal origins of the delay adjustment signals output from the delay adjustment unit 11 when the delay adjustment signals are arranged in all or a portion of each time period. When it is desired to reduce the separation target signal, components having a low spatial frequency may be reduced. When it is desired to obtain the separation target signal only, components mainly having a low spatial frequency may be extracted.

A predetermined threshold of a spatial frequency when signals having a predetermined spatial frequency or lower are reduced may be stored in advance, and the threshold may be determined based on predetermined rules each time as necessary.

In order to perform the process of the spatial frequency filter 12, it is necessary to arrange signals. Thus, when the signals are not arranged by the signal arrangement unit 18, the signals are arranged before the spatial frequency filter 12 performs the processing.

Examples of the spatial frequency filter include a FIR filer, an IIR filter, a moving average filter, and a Gauss filter. However, an optional filter may be used as long as the filter can separate low spatial frequency components. A cutoff frequency of the spatial frequency filter is preferably determined in advance according to the intensity characteristics of the separation target signal. The spatial frequency filter 12 may convert the signals to spatial frequency signals and may reduce frequency components having a predetermined spatial frequency or lower.

If the separation target signal has the same intensity at all measurement positions, only the DC components having the lowest frequency may be separated and reduced. When the intensity of the separation target signal has a positional dependence and is not the same at all measurement positions, the cutoff frequency is set to be higher than a spatial frequency of intensity variations of the separation target signal depending on the measurement position. Moreover, the cutoff frequency may be designated by the user each time, may be determined for each apparatus based on test measurement performed in advance, and may be determined adaptively based on the characteristics of the separation target signal.

(Delay Restoring Unit)

The delay restoring unit 13 performs a reverse process of restoring the delay adjustment performed by the delay adjustment unit 11 on the signals in which the in-phase signals are separated and reduced and which are output by the spatial frequency filter 12. In this way, signals mainly having the same shape as the delay profile obtained by the delay acquiring unit 7 can be separated and reduced. The delay restoring unit corresponds to a phase restoring unit according to the present invention.

(Imaging Processing Unit)

The imaging processing unit 14 serving as an acquirer reconstructs the signals at a plurality of measurement positions obtained by the data processing unit 10 to acquire image data indicating a spatial distribution of signal generation sources. The image obtained herein is an initial acoustic pressure distribution indicating a spatial distribution of an acoustic pressure generated from the light absorber that absorbs light. As a method of the reconstructing process, a universal back-projection method of projecting differentiated signals in a backward direction from the acquisition positions so that the signals overlap each other is preferred. However, other methods can be used as long as the methods can image a spatial distribution of signal generation sources.

In the present embodiment, although signals obtained by separating and reducing the reflection signal are imaged, the imaging processing unit 14 of the present invention is not essential, but the signals obtained by separating and reducing the reflection signal may be displayed. In this case, although it is preferable to display a plurality of arranged signals, only one signal may be displayed. In this way, the user can easily understand the location of the reflection signal and effectively analyze the behavior of reflection.

When the reflection signal is separated, although the plurality of separated signals is imaged, only a portion of the separated signals may be imaged depending on the purpose and the imaging of the reflection signal.

The signal arrangement unit 18, the surface shape acquisition unit 8, the reflection signal estimator 9, the data processing unit 10, the delay adjustment unit 11, the spatial frequency filter 12, the delay restoring unit 13, and the imaging processing unit 14 are formed of a computer having devices such as a CPU or a GPU or circuits such as FPGA or ASIC. Moreover, the respective units may be formed of one device or circuit and may be formed of a plurality of devices or circuits. Moreover, the respective processes performed by the respective units may be executed by any device or circuit. Further, the respective units may share the device or circuit.

(Display Unit)

The display unit 15 displays the results of processing. Specifically, the display unit 15 is a display. Due to this, the user can visually perceive the information on the inside of the object.

(Processing Flow)

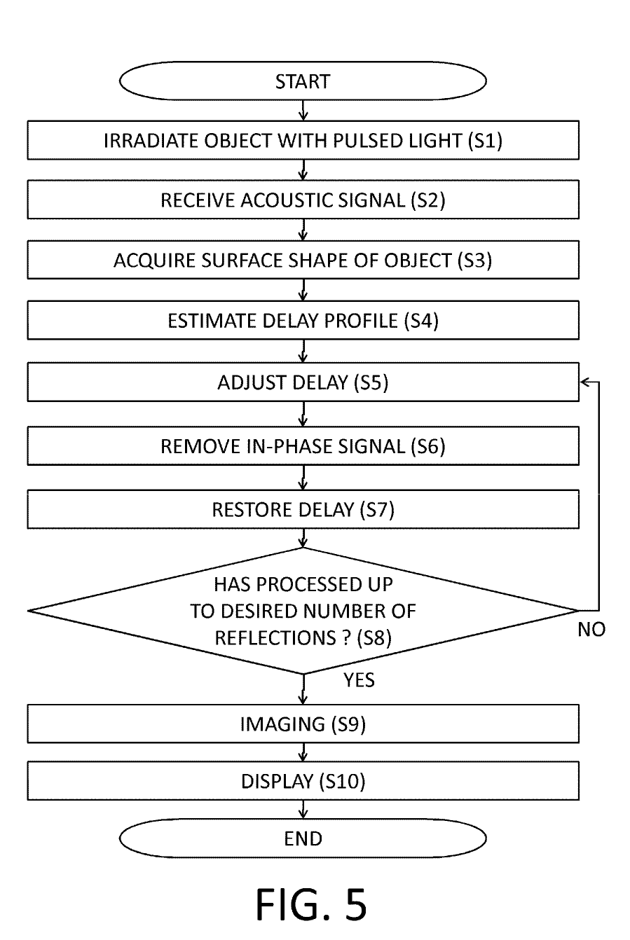

Next, the implementation method of the present embodiment will be described with reference to the flowchart of FIG. 5.

First, an object is irradiated with a pulsating beam (S1), and an acoustic wave generated inside the object is received at a plurality of measurement positions (S2). The acoustic waves received at the respective measurement positions are output as a plurality of time-series reception signals. The surface shape of the object is acquired from the received signal (S3). Moreover, the delay profile of the reflection signal is estimated based on the surface shape (S4). In this way, it is possible to acquire a phase pattern of a target signal to be reduced, included in the plurality of time-series reception signals.

Here, since at least one delay profiles are obtained, the process of S5 to S7 is performed sequentially on the respective delay profiles. First, the delay of the obtained signal is adjusted using a certain delay profile (S5). Moreover, in-phase signals are reduced using a spatial filter (S6). Further, the delay is restored to a value before adjustment (S7).

In S5, adjustment is performed so as to decrease a phase difference. According to a typical example of such adjustment, phases are aligned to occur at the same point in time. However, the phase adjustment method is not limited to the method of aligning the phases so as to occur at the same point in time. That is, the present invention can be realized by such phase adjustment that a phase pattern of the target signal corresponds to a specific spatial direction. When the phase pattern is aligned in the specific spatial direction in this manner, it is possible to remove or reduce the target signal by reducing low-frequency components in the specific spatial direction.

Moreover, although the delay is restored to the original value in S7, this process is not essential. That is, when an image is reconstructed using the signal obtained by reducing the target signal, it is possible to image the characteristic information without restoring the delay by performing calculation by taking the adjusted phase difference into consideration. Specifically, the delay changed by the adjustment may be added to the delay amount applied to when reconstructing a certain target voxel (or pixel).

It is determined whether the processes of S5 to S7 have been performed on all delay profiles corresponding to a desired number of reflections (S8). When the processes have not been completed for all delay profiles, the flow returns to S5. When the processes have been completed for all delay profiles, imaging is performed using the processed signals (S9) and images are displayed (S10).

According to the apparatus of the present embodiment, it is possible to separate and reduce the reflection signal easily and to obtain an image in which artifacts associated with reflection are reduced.

### Modification

In the above-described flow, delay adjustment (S5) based on the phase pattern is performed on the respective delay profiles, and then, the filtering process (S6) is performed. However, the present invention can be realized by processing signals based on the arrangement direction of the target signal among the reception signals without performing the delay adjustment. That is, if the arrangement direction of the target signal in the plurality of time-series reception signals is known, an operation of reducing low-frequency components in the arrangement direction can be realized easily. As a result, a signal in which low-frequency components corresponding to artifacts are reduced is obtained. By acquiring the characteristic information using this signal, it is possible to reconstruct an image in which artifacts are reduced.

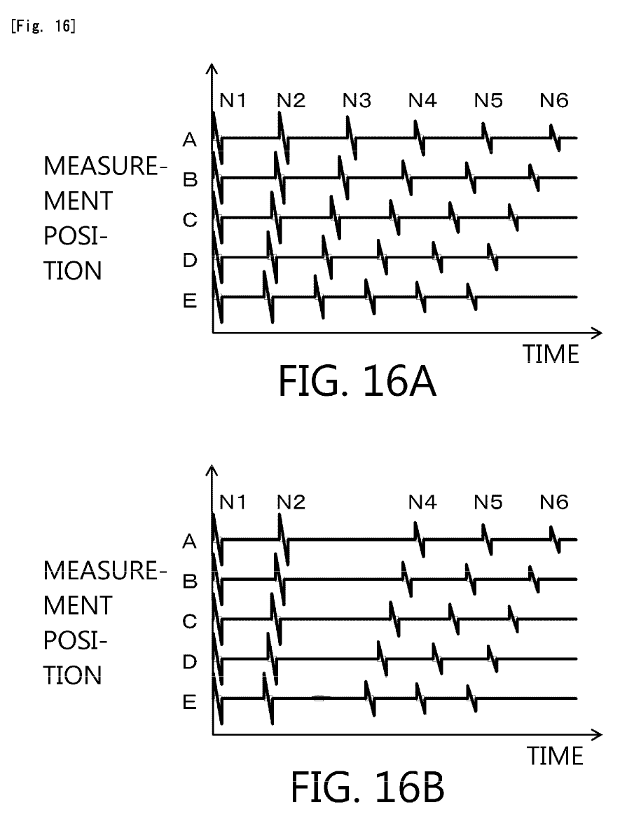

The concept of this modification will be described with reference to FIGS. 16A and 16B. FIG. 16A illustrates a state where target signals generated by multiple reflection are included in a reception signal similarly to FIG. 3A. For example, in sequence N3, the target signal is arranged from the top-right corner to the bottom-left corner. Thus, by performing Fourier transform in the spatial direction (the arrangement direction of the target signal) based on the arrangement information of the target signal to remove low-frequency components, it is possible to reduce components derived from multiple reflection.

When information on the arrangement direction (phase pattern) is acquired, a delay acquiring unit may be used and an arrangement direction acquisition unit provided separately may be used. For example, the arrangement of the target signal can be acquired based on at least one of the coordinate information of the outer shape of an object and the coordinate information of a plurality of measurement positions. Moreover, the arrangement of the target signal can be acquired by performing calculation using the surface shape of the object and the positional relation of the plurality of measurement positions. For example, the arrangement direction acquisition unit may include a known three-dimensional camera or the like for acquiring the coordinate information of the outer shape of the object.

### <Second Embodiment> (Case where Separation Target Signal is not Reflection Wave, Delay Profile is Prepared in Advance or Manually Designated)

In the present embodiment, a case where the delay profile is prepared in advance or is input by the user will be described.

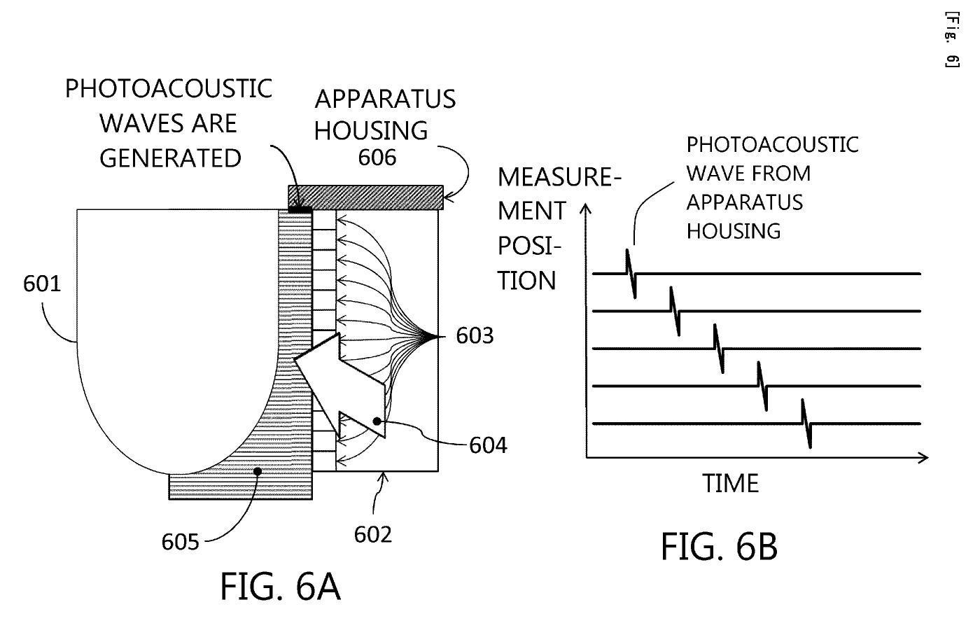

As illustrated in FIG. 6A, a case where photoacoustic waves are generated from an apparatus housing 606 of which the relative position to an acoustic detector 602 is always the same will be considered. In FIGS. 6A and 6B, a plurality of acoustic detection devices 603 included in the acoustic detector 602 receives photoacoustic waves from an object 601 irradiated with a pulsating beam 604 with an acoustic matching member 605 interposed. A signal obtained in this case is as illustrated in FIG. 6B.

As illustrated in FIG. 6B, the photoacoustic waves generated from the apparatus housing 606 appear always at the same position regardless of measurement. However, since the pulsating beam reflected from the object is also absorbed by the apparatus housing, it is thought that the intensity of the photoacoustic wave generated from the apparatus housing is different from object to object. In such a case, if the position at which peaks appear are always the same, it is possible to reduce signals at the appearance positions using the apparatus of the present invention by specifying the appearance positions based on calculations or measurements performed in advance. In this method, it is possible to designate the delay profile easily.

(Apparatus Configuration)

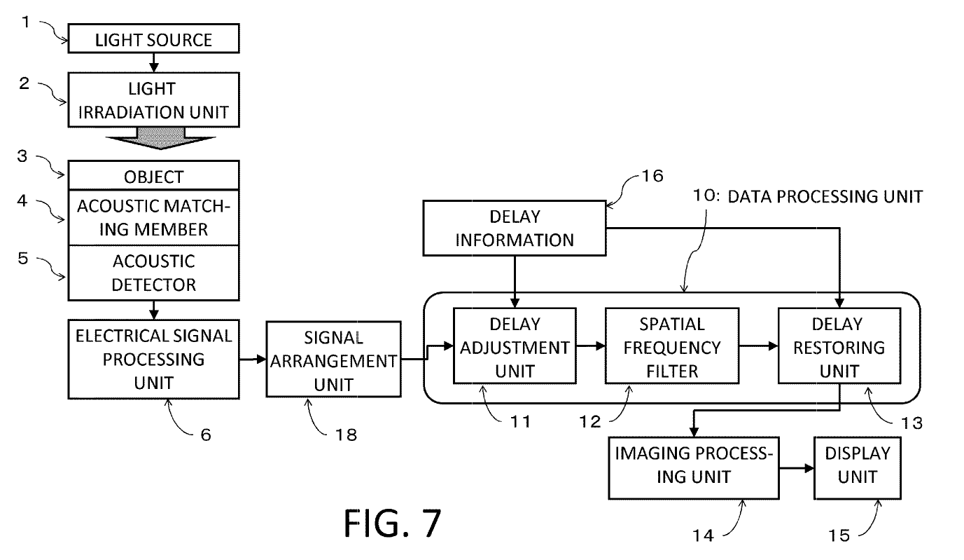

Constituent components of the present embodiment are illustrated in FIG. 7. Unlike the first embodiment, delay information 16 is used instead of the delay acquiring unit 7. The delay information 16 is the delay profile of a separation target signal, obtained based on calculations or measurements performed in advance. Specifically, the delay information is stored in a storage medium or a storage device or is stored in an external device via a signal line or a network.

As a method of acquiring the delay information in advance, it is preferable to measure a plurality of phantoms including different light absorbers and extract common signals as separation target signals to obtain the delay profile. Moreover, measurements may be performed using a phantom that does not include a light absorber to observe signals, and the signals may be extracted as separation target signals to obtain the delay profile. Further, an apparatus arrangement may be reflected on an acoustic propagation simulation to simulate signals when photoacoustic waves are generated from the apparatus housing to obtain the delay profile.

(Another Apparatus Configuration)

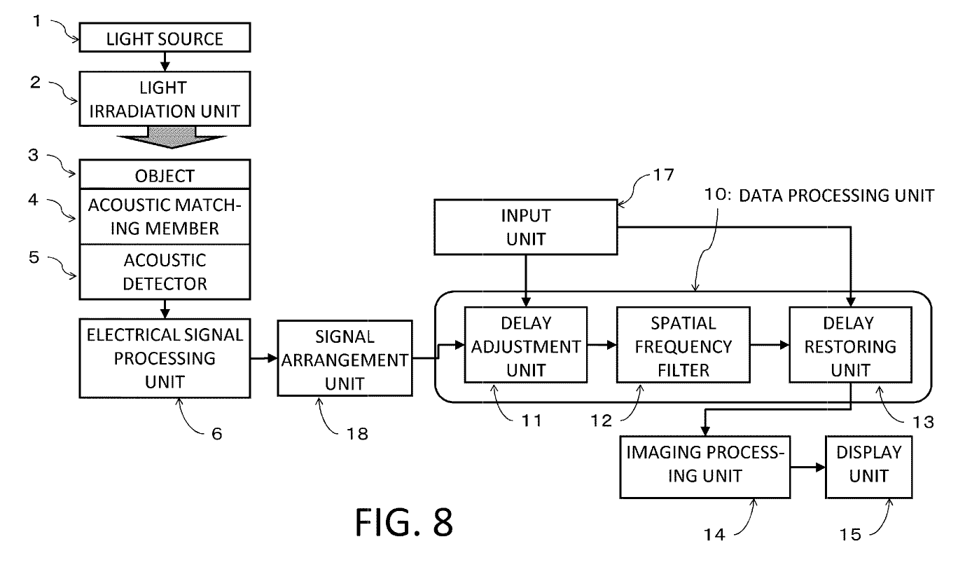

As another method of obtaining the delay profile, a value designated by the user may be used. For example, when the sound speed changes due to the influence of temperature, the appearance positions or the delays are generally the same. In this example, a case where the appearance positions or the delays vary to some extent and are not exactly identical in the respective measurements will be considered. In such a case, the separation target signals can be reduced by an apparatus including such constituent components as illustrated in FIG. 8. Unlike the first embodiment, an input unit 17 is used instead of the delay acquiring unit 7.

With the input unit 17, the user inputs the delay profile of a desired separation target signal. The input unit 17 is an input device such as a mouse or a keyboard and preferably includes a display device such as a display with which the user can monitor input results and signals.

As an input method, it is preferable to input and designate a line following the delay profile of a desired separation target signal using a mouse. Alternatively, the numerical values of the coordinates may be input using a keyboard. Further, an input delay profile may be used as an initial value and may be fitted to a signal having a high intensity. In this way, it is possible to relieve the load on the user. In this case, enhancement processing such as noise reduction or template matching may be performed so that a desired separation target signal is emphasized.

As described above, various methods of obtaining the separation target signal and the delay profile of the separation target signal can be considered. However, the scope of the present invention is not limited by the method of obtaining the delay profile. According to the present invention, it is possible to separate and reduce separation target signals.

### <Third Embodiment> (Bowl Shape)

In the present embodiment, a case where the acoustic detector is not planar will be described.

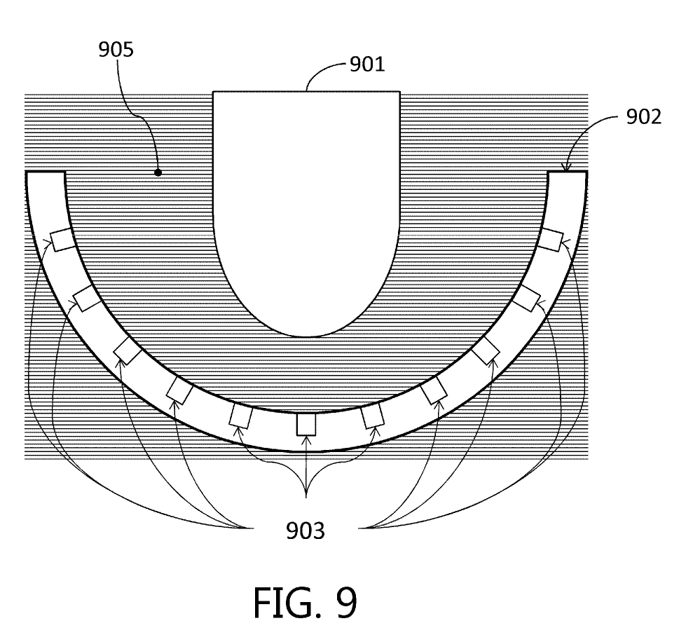

In FIG. 9, a plurality of acoustic detection devices 903 included in an acoustic detector 902 receives photoacoustic waves from an object 901 irradiated with a pulsating beam with an acoustic matching member 905 interposed.

As illustrated in FIG. 9, it is assumed that the acoustic detector has a curved surface and reflection waves reflected within the acoustic matching member among the photoacoustic waves generated from the object surface are separation target signals. In this case, similarly to the respective embodiments, the separation target signals can be separated and reduced by arranging the signals at respective measurement positions to adjust the delay, separating and reducing in-phase signals using a filter, and restoring the delay to an original value.

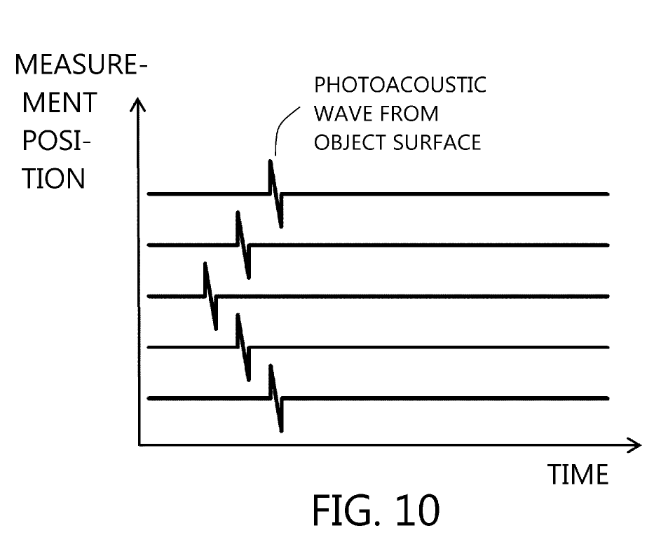

FIG. 10 illustrates the obtained signals arranged so that the temporal origins forma flat surface. In this manner, the arrangement of signals may not be identical to the spatial arrangement of actual storage devices. The subsequent processes are performed similarly to the first and second embodiment, whereby the separation target signals can be separated and reduced.

### <Fourth Embodiment> (Case where Intensity of Separation Target Signal Varies)

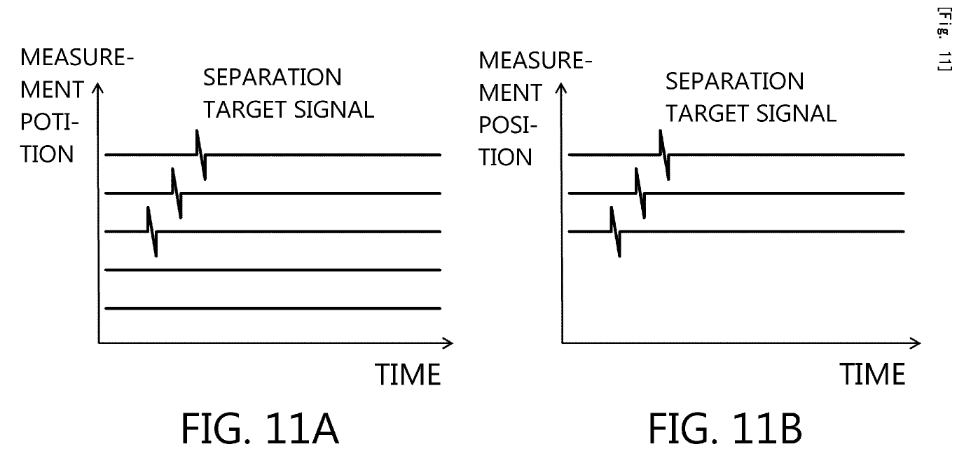

Depending on a positional relation between the acoustic detector and the acoustic generation source, separation target signals may appear at some measurement positions only as illustrated in FIGS. 11A and 11B. In this case, even when the delay of only the measurement positions where the separation target signals are present is adjusted and the low-frequency components in the arrangement direction are separated by the spatial frequency filter 12, the low-frequency components are not separated satisfactorily if the intensity changes abruptly at a certain position. In the present embodiment, a case where the intensity of the separation target signal changes greatly depending on the measurement position will be described.

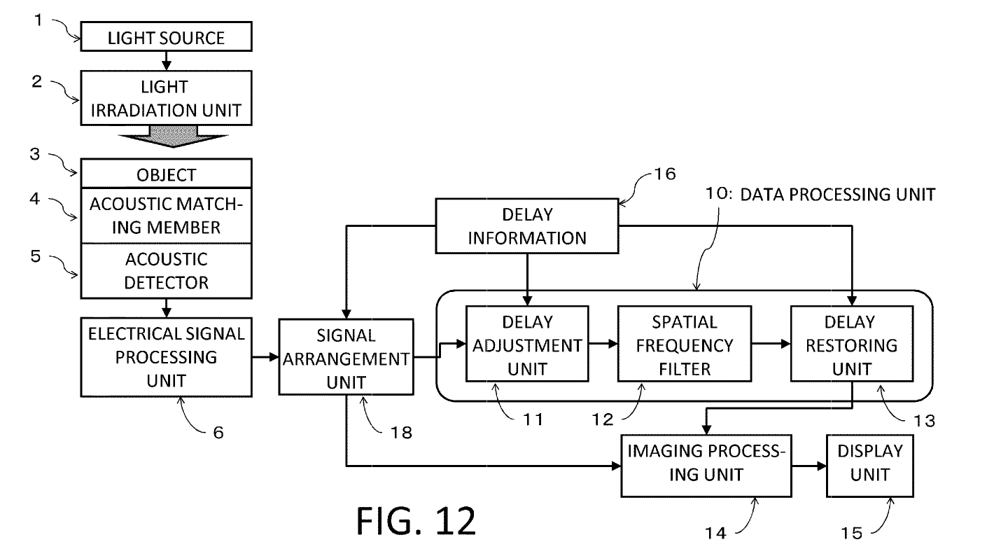

The constituent components of the present embodiment are as illustrated in FIG. 12. Delay information 16 has such information that a measurement position where the separation target signal is present has information on a delay profile and a measurement position where the separation target signal is not present does not have any information on the delay profile. Specifically, the delay information 16 is information stored in an optional storage medium or the like.

In this case, the signal arrangement unit 18 regards only the measurement positions where the separation target signal is present as a processing target and masks signals at measurement positions where the separation target signal is not present by regarding the same as a non-processing target. Due to this, the data processing unit 10 treats the signals of FIG. 11A virtually as being those of FIG. 11B. As a result, the position where the intensities of the separation target signals are different are eliminated, and the spatial frequency filter 12 can separate and reduce the separation target signals. After the data processing unit 10 finishes the processing, non-processing target signals and the processed signals are imaged by the imaging processing unit 14.

Even when the signal arrangement unit 18 is not present, the same results can be obtained by the delay adjustment unit 11, the spatial frequency filter 12, and the delay restoring unit 13 performing processing while masking the non-processing target signals. Moreover, processing targets and non-processing targets may be manually input, and a threshold may be determined so that those equal to or higher than the threshold among the separation target signals may be regarded as the processing targets.

### Test Example

The effects of the present invention were verified from tests.

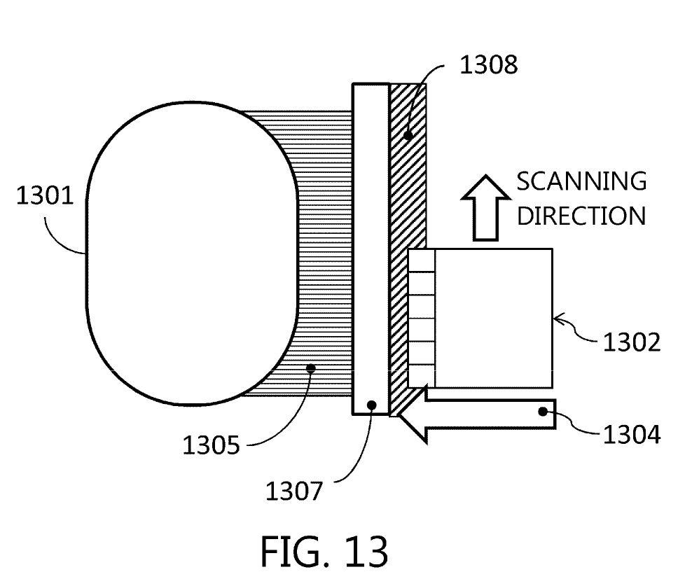

In FIG. 13, a plurality of acoustic detection devices included in an acoustic detector 1302 receives photoacoustic waves generated and propagated when an object 1301 is irradiated with a pulsating beam 1304 with an acoustic matching member 1305, an object holding plate 1307, and an acoustic matching liquid 1308 interposed.

In the test system illustrated in FIG. 13, an object was the calf of a living body, and a gel-shaped acoustic matching member was provided in contact with the object. The acoustic matching member was made of a flexible material and was fit to the shape of the living body. Moreover, a 7 mm-thick object holding plate formed from polymethylpentene was provided. Further, an acoustic matching liquid which is castor oil was filled in a 3 mm-thick space between the acoustic detector and the object holding plate. Both surfaces of the object holding plate were parallel to the acoustic matching liquid.

The acoustic detector and the pulsating beam were moved in synchronization for scanning so that all regions being in contact with the object were measured. A PZT of which the diameter of a receiving unit was 2 mm and of which a bandwidth was 80% at a central frequency of 1 MHz was used as the device of the acoustic detector. 15*23 devices were arranged in a planar direction to form one acoustic detector. A TiS laser that generates a pulsating beam having a wavelength of 797 nm and a pulse width of several nanoseconds was used as the light source of the pulsating beam.

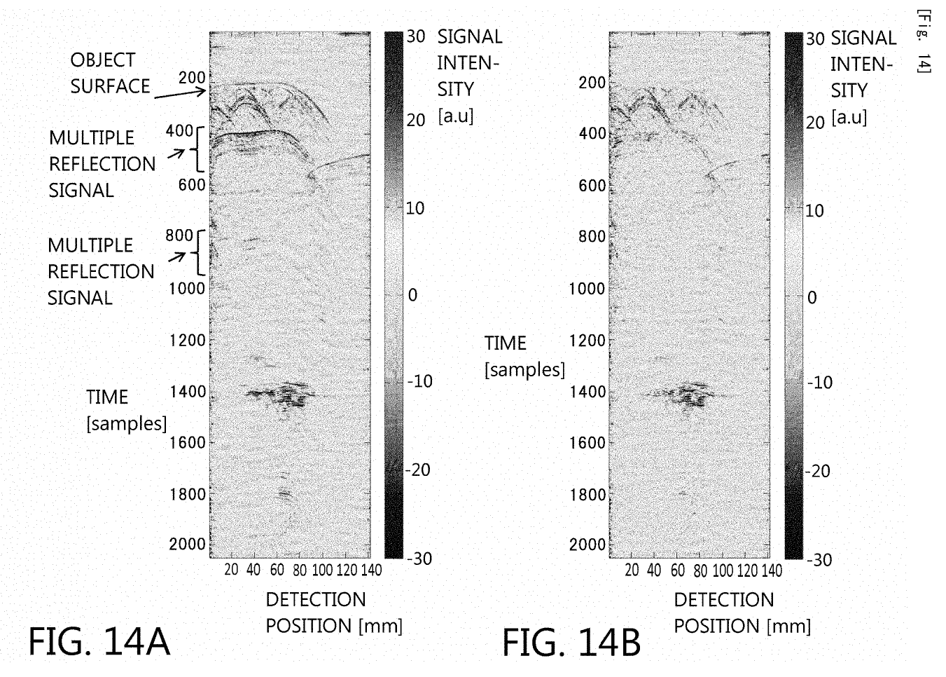

In this test system, irradiation of pulsating beams, collection of acoustic signals, and scanning were performed repeatedly to obtain all pieces of signal data. In this case, an analog-digital converter having a sampling frequency of 20 MHz and a resolution of 12 bit was used. FIG. 14A illustrates the obtained signals arranged in conformity with the measurement positions.

In FIG. 14A, the object surface was observed at the position of 200 samples and this shape is the delay profile of the object surface. After that, a group of multiple-reflection signals appeared at the positions of 400 to 600 samples. The reason why a plurality of reflection signals rather than one reflection signal appears is because there is a plurality of multiple-reflection layers and reflections occur at different intervals. Moreover, a group of multiple-reflection signals also appeared at the positions of 800 to 100 samples. In this region, reflections repeat and the signal intensity decreases.

FIG. 14B illustrates multiple-reflection signals which are reduced using the apparatus described in the second embodiment. In this example, the number of delay profiles of the reflection signal to be estimated was four. The reflection signal was reduced using the shapes obtained by delaying the delay profile of the object surface in the time direction by zero, one, two, and three times as the delay profiles of the reflection signal. When FIGS. 14A and 14B are compared, it can be understood that the multiple-reflection signal is reduced.

Subsequently, the signal was imaged and processed to obtain a 3-dimensional image. Universal back-projection was used for the imaging.

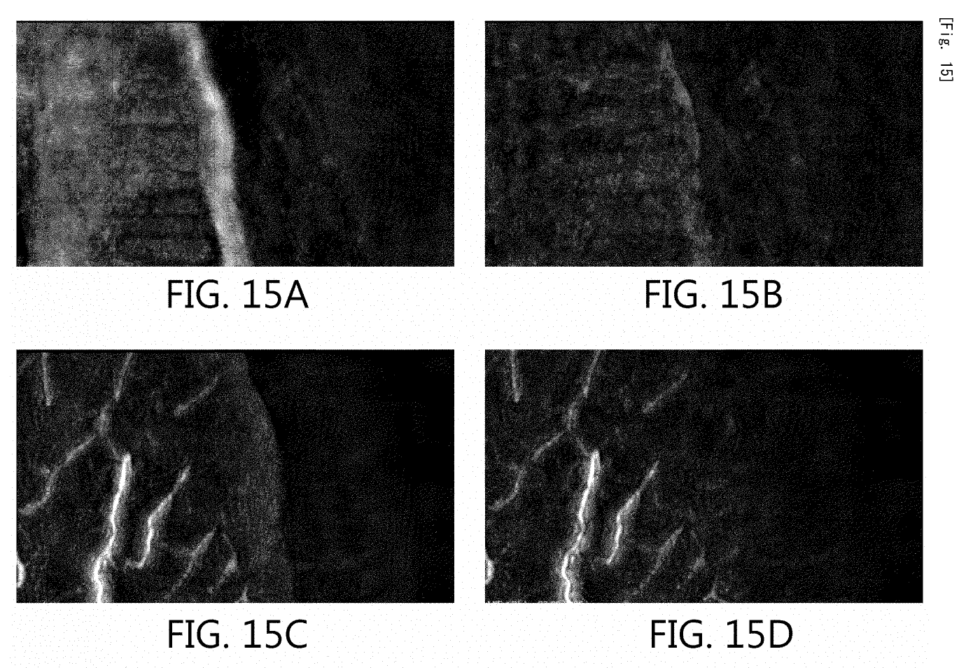

FIG. 15A illustrates an image obtained by imaging the non-processed signal illustrated in FIG. 14A and displaying the slice of the reflection signal. FIG. 15B illustrates an image obtained by imaging the signal processed using the apparatus described in the second embodiment, illustrated in FIG. 14B and displaying the same slice as FIG. 15A. According to the comparison between both images, when the signal is not processed, the reflection signal reflecting the surface shape of the object is imaged to appear as artifacts. However, when the reflection signal is reduced using the apparatus of the present invention, artifacts are reduced.

Moreover, FIGS. 15C and 15D illustrate 3-dimensional images created from non-processed signals and processed signals in the slice in which a structure derived from a living body appears remarkably. It can be understood that the structure derived from the living body is rarely influenced by the processing.

From the above, it was confirmed that by using the apparatus of the present embodiment, it is possible to reduce artifacts mainly without having a significant influence on the structure derived from the living body.

### Other Embodiments

Embodiments of the present invention can also be realized by a computer of a system or apparatus that reads out and executes computer executable instructions recorded on a storage medium (e.g., non-transitory computer-readable storage medium) to perform the functions of one or more of the above-described embodiment(s) of the present invention, and by a method performed by the computer of the system or apparatus by, for example, reading out and executing the computer executable instructions from the storage medium to perform the functions of one or more of the above-described embodiment(s). The computer may comprise one or more of a central processing unit (CPU), micro processing unit (MPU), or other circuitry, and may include a network of separate computers or separate computer processors. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2014-023285, filed on Feb. 10, 2014, and Japanese Patent Application No. 2015-006471, filed on Jan. 16, 2015, which are hereby incorporated by reference herein in their entirety.

1. A photoacoustic apparatus comprising:
a receiving unit configured to receive acoustic waves at a plurality of measurement positions and convert the acoustic waves to a plurality of time-series reception signals; and
a processing unit configured to (a) adjust phases of the plurality of time-series reception signals so that a phase pattern of a target signal among the plurality of time-series reception signals corresponds to a specific spatial direction, (b) reduce low-frequency components in the specific spatial direction from the plurality of time-series reception signals of which the phases have been adjusted, and (c) acquire characteristic information of an object based on the plurality of time-series reception signals of which the low-frequency components have been reduced.

2. The photoacoustic apparatus according to claim 1, wherein the processing unit performs the adjustment so that a phase difference of the target signal among the plurality of time-series reception signals decreases.

3. The photoacoustic apparatus according to claim 1, wherein the processing unit performs the adjustment so that a phase of the target signal is aligned in an arrangement direction of the plurality of measurement positions.

4. The photoacoustic apparatus according to claim 1, wherein after reducing the low-frequency components and before acquiring the characteristic information of the object, the processing unit restores the phases of the plurality of time-series reception signals to the phases before the adjustment.

5. The photoacoustic apparatus according to claim 1, wherein the processing unit performs an operation based on the phase difference adjusted for each of the plurality of time-series reception signals when acquiring the characteristic information of the object.

6. The photoacoustic apparatus according to claim 1, further comprising:
a delay acquiring unit configured to acquire a phase pattern of the target signal among the plurality of time-series reception signals.

7. The photoacoustic apparatus according to claim 6, wherein the delay acquiring unit acquires the phase pattern based on at least one of a surface shape of the object and the coordinates of the plurality of measurement positions.

8. The photoacoustic apparatus according to claim 1, further comprising:
an input unit configured to allow a user to input the target signal, wherein the processing unit adjusts the phases of the plurality of time-series reception signals based on an input from the input unit.

9. The photoacoustic apparatus according to claim 8, wherein
the processing unit displays the plurality of time-series reception signals on a display unit, and
the input unit is configured to be able to input the target signal from the time-series reception signals displayed on the display unit.

10. The photoacoustic apparatus according to claim 1, wherein the processing unit reduces components of a signal based on an acoustic wave reflected from a surface of the object, as the target signal.

11. The photoacoustic apparatus according to claim 1, wherein the processing unit reduces components of a signal based on an acoustic wave reflected from the receiving unit, as the target signal.

12. The photoacoustic apparatus according to claim 1, wherein
the receiving unit is in contact with the object with an acoustic matching member interposed, and
the processing unit reduces components of a signal based on a reflection wave reflected from an interface of the acoustic matching member, as the target signal.

13. The photoacoustic apparatus according to claim 12, wherein the processing unit reduces components of a signal based on a reflection wave having undergone multiple reflection from an interface of the acoustic matching member, as the target signal.

14. The photoacoustic apparatus according to claim 1, wherein the processing unit performs processing while masking a signal acquired at some of the measurement positions.

15. The photoacoustic apparatus according to claim 1, wherein the processing unit acquires a spatial frequency signal from the phase-adjusted signal using a spatial frequency filter and reduces a signal having a predetermined spatial frequency or lower from the spatial frequency signal.

16. A photoacoustic apparatus comprising:
a receiving unit configured to receive acoustic waves at a plurality of measurement positions and convert the acoustic waves to a plurality of time-series reception signals; and
a processing unit configured to (a) acquire information on an arrangement direction of a target signal among the plurality of time-series reception signals, (b) reduce low-frequency components in the arrangement direction of the target signal from the plurality of time-series reception signals based on the information on the arrangement direction of the target signal, and (c) acquire characteristic information on an inside of an object based on the plurality of time-series reception signals of which the low-frequency components have been reduced.

17. A signal processing method of acquiring characteristic information of an object based on a plurality of time-series reception signals obtained by receiving acoustic waves at a plurality of measurement positions, comprising:
adjusting phases of the plurality of time-series reception signals so that a phase pattern of a target signal among the plurality of time-series reception signals corresponds to a specific spatial direction;
reducing low-frequency components in the specific spatial direction from the plurality of time-series reception signals of which the phases have been adjusted; and
acquiring characteristic information of an object based on the plurality of time-series reception signals of which the low-frequency components have been reduced.

18. A signal processing method of acquiring characteristic information of an object based on a plurality of time-series reception signals obtained by receiving acoustic waves at a plurality of measurement positions, comprising:
acquiring information on an arrangement direction of a target signal among the plurality of time-series reception signals;
reducing low-frequency components in the arrangement direction of the target signal from the plurality of time-series reception signals based on the information on the arrangement direction of the target signal; and
acquiring characteristic information of an inside of an object based on the plurality of time-series reception signals of which the low-frequency components have been reduced.
